(19) United States
(12) Patent Application Publication
(10) Pub. No.: US 2023/0034390 A1
(43) Pub. Date: Feb. 2, 2023

(54) HIGH PERFORMANCE LIQUID CHROMATOGRAPHY QUANTIFICATION OF EXCIPIENTS

(71) Applicant: Seagen Inc., .Bothell, WA (US)

(72) Inventors: Jing Liu, Bothell, WA (US); Ethan Tyler Brickman, Bothell, WA (US); Luke David Munier Cody, Seattle, WA (US)

(73) Assignees: Seagen Inc., Bothell, WA (US); Seagen Inc., Bothell, WA (US)

(21) Appl. No.: 17/786,107

(22) PCT Filed: Dec. 15, 2020
(86) PCT No.: PCT/US2020/065162

§ 371 Date: Jun. 16, 2022

Related U.S. Application Data
(60) Provisional application No. 62/948,357, filed on Dec. 16, 2019.

(51) Int. Cl. G01N 30/88; G01N 30/62; G01N 33/15

(52) U.S. Cl. CPC G01N 30/88; G01N 30/62; G01N 33/15; G01N 2030/8836; G01N 2030/027

(57) ABSTRACT

The present invention provides an analytical method for separating and optionally quantifying two or more buffers or excipients in a sample in a single assay.

### CROSS-REFERENCE TO RELATED APPLICATIONS

This application claims the benefit of and priority to U.S. Provisional Patent Application No. 62/948,357, filed Dec. 16, 2019, the disclosure of which is hereby incorporated by reference in its entirety for all purposes.

### BACKGROUND

High Performance Liquid Chromatography (HPLC) is a commonly-used analytic method for separation and quantification of liquid sample components. Sample components eluted from an HPLC column pass through a detection cell and are detected using any one or more than one method including, but not limited to, ultraviolet detection, mass spectrometry, refractive index detection, evaporative light scattering detection (ELSD), charged aerosol detection (CAD), and condensation nucleation light scattering detection (CNLSD).

Pharmaceutical formulations routinely comprise one or more than one active ingredient in combination with a plurality of physiologically-acceptable excipients and/or carriers. These excipients and/or carriers may contribute to improving stabilization, dilution or bulking, promoting absorption, reducing viscosity, and/or enhancing solubility of the active ingredient. Preclinical and clinical studies often require an analysis of the physicochemical properties of a pharmaceutical formulation preparation, which invariably includes quantification of the excipients and/or carriers used therein. As the physicochemical properties of constituent excipients can vary widely, multiple analyses by HPLC using multiple columns packed with different stationary phases are frequently employed.

There is a need for improved methods of separating, detecting, and quantifying two or more excipients in a pharmaceutical formulation having different physicochemical characteristics, such that the improved method is able to resolve and detect the two or more excipients in a single HPLC run using a single stationary phase column.

### SUMMARY OF THE INVENTION

The present invention generally relates to a method for analytically separating and optionally quantifying two or more buffers or excipients in a single HPLC assay.

In one aspect, the method of the present invention comprises: performing chromatography on a test sample comprising two or more buffers or excipients, on a pentafluorophenyl (PFP) high performance liquid chromatography (HPLC) column to separate the two or more buffers or excipients; detecting the two or more separated buffers or excipients in the HPLC column effluent; and generating a chromatogram having peaks corresponding to the separated two or more buffers or excipients.

In some embodiments, the two or more buffers or excipients present in the test sample are selected from sodium phosphate, sodium citrate, potassium phosphate, histidine, and sugars or sugar based molecules. For example, in some embodiments the sugars or sugar based molecules are selected from 2-hydroxypropyl-beta-cyclodextrin (hpβCD), sucrose, trehalose, and mannitol.

In some embodiments, the method of the present invention further comprises: obtaining standard calibration chromatographic data for the two or more excipients run on the same HPLC column; and calculating a concentration or an amount of the two or more buffers or excipients in the test sample by determining from the chromatogram integrated peak areas of the two or more buffers or excipients and converting the integrated areas to the concentration or amount based on the obtained standard calibration chromatographic data. In certain embodiments, the conversion includes a linear regression fit to the standard calibration chromatographic data.

In some embodiments, the two or more buffers or excipients in the test sample are detected using an evaporative light scattering detector (ELSD). In certain embodiments the ELSD is set at an evaporative temperature of 40 to 70° C., a pressure of 30 to 70 psi, a gain of 0.5 to 2, and filter set at 0.5 to 1.

In other embodiments, the two or more buffers or excipients in the test sample are detected using a charged aerosol detector (CAD). In certain embodiments the CAD is set at an evaporative temperature of 25 to 35° C., a frequency of 4 to 6 Hz, a filter set at 4 to 6 seconds, a power function set to 1.78 for the first two-thirds of an HPLC run, and a power function set to 1.68 for the last third of the HPLC run.

In some embodiments of the present invention, the HPLC is run using two mobile phases: mobile phase A and mobile phase B. In some embodiments, mobile phase A is 100% H₂O. In other embodiments, mobile phase A comprises H₂O and formic acid. In still other embodiments, mobile phase A comprises H₂O, and trifluoroacetic acid. In some embodiments, the mobile phase B comprises acetonitrile.

In some embodiments mobile phase A is H₂O and 0.5% formic acid. In other embodiments, mobile phase A comprises H₂O and 0.05% trifluoroacetic acid. In some embodiments, mobile phase B is 100% acetonitrile.

In some embodiments, performing chromatography comprises an equilibration step having a flow of 100% mobile phase A through the HPLC column at a rate of 0.1 ml/minute to 1.0 ml/minute. In some embodiments, the equilibration step flow rate is 0.25 ml/minute or 0.5 ml/min. In some embodiments, the equilibration step is between 0.5 minutes and 10 minutes. In certain embodiments, the equilibration step is 3.0 minutes or 4.0 minutes.

In some embodiments, performing chromatography comprises a gradient change flow of 60% mobile phase A and 40% mobile phase B through the HPLC column. In some embodiments, performing chromatography comprises a gradient change flow of 40% mobile phase A and 60% mobile phase B through the HPLC column. In some embodiments, performing chromatography comprises a gradient change flow of 100% mobile phase A through the HPLC column. In some embodiments the gradient change flow rate is between 0.1 ml/minute to 1.0 ml/minute. In certain embodiments, the gradient change flow rate is 0.25 ml/minute or 0.5 ml/minute. In some embodiments, the gradient change is between 0.5 minutes and 10 minutes. In certain embodiments, the gradient change is 0.5 minutes, 2.0 minutes, or 4.0 minutes.

In some embodiments, performing chromatography comprises a maintenance step flow of 40% mobile phase A and 60% mobile phase B through the HPLC column. In some embodiments, performing chromatography comprises a maintenance step flow of 100% mobile phase A through the HPLC column. In some embodiments, the maintenance step flow rate is between 0.1 ml/minute and 1.0 ml/minute. In certain embodiments, the maintenance step flow rate is 0.5 ml/minute or 1.0 ml/minute. In some embodiments, the maintenance step is between 0.5 minutes and 10 minutes. In certain embodiments, the maintenance step is 2.5 minutes or 4.0 minutes.

In some embodiments, performing chromatography comprises a re-equilibration step having a flow of 100% mobile phase A through the HPLC column at a rate of 0.1 ml/minute to 1.0 ml/minute. In certain embodiments, the re-equilibration flow rate is 0.25 ml/minute or 0.5 ml/minute. In some embodiments, the re-equilibration step is between 0.5 minutes and 10 minutes. In certain embodiments, the re-equilibration step is 3.0 minutes.

In some embodiments of the present invention, performing chromatography comprises: (i) equilibration with 100% mobile phase A at a flow rate of 0.25 ml/minute for 3.0 minutes; (ii) gradient change to 60% mobile phase A and 40% mobile phase B at a flow rate of 0.25 ml/minute for 0.5 minutes; (iii) gradient change to 40% mobile phase A and 60% mobile phase B at a flow rate of 1.0 ml/minute for 4.0 minutes; (iv) maintenance at 40% mobile phase A and 60% mobile phase B at a flow rate of 1.0 ml/minute for 2.5 minutes; (v) gradient change to 100% mobile phase A at a flow rate of 1.0 ml/minute for 2 minutes; and (vi) re-equilibration with 100% mobile phase A at a flow rate of 0.25 ml/minute for 3.0 minutes.

In other embodiments of the present invention, performing chromatography comprises: (i) equilibration with 100% mobile phase A at a flow rate of 0.5 ml/minute for 4.0 minutes; (ii) gradient change to 40% mobile phase A and 60% mobile phase B at a flow rate of 0.5 ml/minute for 2.0 minutes; (iii) gradient change to 100% mobile phase A at a flow rate of 0.5 ml/minute for 2.0 minutes; and (iv) maintenance at 100% mobile phase A at a flow rate of 0.5 ml/minute for 4 minutes.

In other embodiments of the present invention, performing chromatography comprises: (i) equilibration with 100% mobile phase A at a flow rate of 0.5 ml/minute for 4.0 minutes; (ii) gradient change to 40% mobile phase A and 60% mobile phase B at a flow rate of 0.5 ml/minute for 2.0 minutes; (iii) gradient change to 100% mobile phase A at a flow rate of 0.5 ml/minute for 2.0 minutes; and (iv) maintenance at 100% mobile phase A at a flow rate of 0.5 ml/minute for 4 minutes.

In some embodiments of the present invention, 1 μl to 100 μl of the test sample is injected into the HPLC column. In certain embodiments, 10 μl or 4 μl of the test sample is injected into the HPLC column.

In certain embodiments of the present invention, the PFP HPLC column is a 2.6 μm 150×4.6 mm column.

### BRIEF DESCRIPTION OF THE DRAWINGS

FIG. 1A shows a chromatogram of a formulation buffer comprising histidine, sucrose and mannitol resolved on a C18 column.

FIG. 1B shows a chromatogram of a formulation buffer comprising tris, sucrose, and hydroxypropyl β cyclodextrin (hpβCD) chromatographed on a C18 column.

FIG. 2A shows a chromatogram of a formulation buffer comprising histidine, sucrose and mannitol chromatographed on an anion-exchange column.

FIG. 2B shows a chromatogram of a formulation buffer comprising Tris, sucrose, and hpβCD resolved on an anion-exchange column.

FIG. 3 shows a chromatogram of a formulation buffer comprising hpβCD and sucrose resolved on a pentafluorophenyl (PFP) column.

FIG. 4 shows a chromatogram of a formulation buffer comprising Tris, resolved on a PFP column.

FIG. 5 shows chromatograms of formulation buffers comprising sodium phosphate (A), sodium citrate (B), potassium phosphate (C), histidine (D), and trehalose (E) resolved on a PFP column.

FIG. 6 shows a chromatogram of a formulation buffer comprising mannitol, sucrose and histidine resolved on a PFP column.

### DETAILED DESCRIPTION

Disclosed herein is a method for analytically separating and optionally quantifying two or more buffers or excipients in a sample in a single assay using a pentafluorophenyl (PFP) high performance liquid chromatography (HPLC) column. This method is advantageous in its ability to resolve two or more buffers or excipients in a sample that conventional methods known in the art would not be able to resolve due to solvent interference and/or sub-optimal column retention times and peak shapes.

To facilitate an understanding of the present invention, a number of terms and phrases are defined below.

The terms “a” and “an” as used herein mean “one or more” and include the plural unless the context is appropriate.

The term “about” as used herein means value at or near a stated amount. For example, “about” can refer to less than or equal to ±5%, such as less than or equal to ±2%, such as less than or equal to ±1%, such as less than or equal to ±0.5%, such as less than or equal to ±0.2%, such as less than or equal to ±0.1%, or such as less than or equal to ±0.05%.

The term “analyte” as used herein means a substance or chemical constituent being identified and/or measured.

The term “series of standard calibration samples” as used herein means two or more samples, each sample having a different, known concentration of analyte, and wherein the range of concentrations of the different samples cover, or is near to, the expected concentration of the analyte in a test sample.

The term “calibration curve” as used herein means a plot based on the analyte signal detected and measured by the HPLC instrument for each known concentration of sample comprising the series of standard calibration samples.

The term “linear regression fit” as used herein means a mathematical algorithm that plots a line in which a set of signal data has a minimal measurement from that line. Plots resulting from a linear regression fit have a slope, y-intercept, and an R-squared value that is a measure of how well the signal data fits the line.

The term “integrated peak areas” as used herein means the quantified areas under the chromatographic peaks corresponding to the detected analyte signals of analytes in a test sample.

The term “mobile phase” as used herein means a liquid or gas that flows through a chromatography instrument, wherein the liquid or gas moves one or more than one analyte in a sample at different rates over a stationary phase.

The term percent (or %), when used in connection with mobile phase descriptions, is expressed as a volume percentage, unless the context clearly dictates otherwise.

The term “stationary phase” as used herein means a solid or liquid in a chromatography instrument on which one or more than one analyte is separated or selectively adsorbed.

### Buffers and Excipients

The present invention provides an analytical method for quantifying two or more buffers or excipients in a sample in a single assay. In some embodiments, the sample may be a pharmaceutical composition comprising two or more buffers or excipients formulated with an effective amount of an active ingredient as described in Remington's Pharmaceutical Sciences, Mack Publishing Company, Philadelphia, Pa., 17th ed., 1985. For example, in some embodiments, the two or more buffers or excipients may be selected from, but not limited to, carbohydrates (e.g., glucose, sucrose, galactose, mannose, ribose, arabinose, xylose, fructose, maltose, cellobiose, lactose, deoxyribose, hexose); sugar-based molecules (e.g., mannitol, sorbitol, ethylene glycol, glycerol, erythritol, threitol, arabitol, xylitol, ribitol, galactilol, fucitol, iditol, inositol, volemitol, lactitol, isomalt, maltitol, maltotriitol, and polyglycitol); cyclodextrins (e.g., α-cyclodextrin, β-cyclodextrin, γ-cyclodextrin, hydroxypropyl βcyclodextrin (hpβCD), and sulfobutylether βcyclodextrin); lipids (e.g. lauric acid (12:0) myristic acid (14:0), palmitic acid (16:0), palmitoleic acid (16: 1), margaric acid (17:0), heptadecenoic acid (17: 1), stearic acid (18:0), oleic acid (18: 1), linoleic acid (18:2), linolenic acid (18:3), octadecatetraenoic acid (18:4), arachidic acid (20:0), eicosenoic acid (20: 1), eicosadienoic acid (20:2), eicosatetraenoic acid (20:4), eicosapentaenoic acid (20:5) (EPA), docosanoic acid (22:0), docosenoic acid (22: 1), docosapentaenoic acid (22:5), docosahexaenoic acid (22:6) (DHA), and tetracosanoic acid (24:0)); minerals (e.g., chloride, sodium, calcium, iron, chromium, copper, iodine, zinc, magnesium, manganese, molybdenum, phosphorus, potassium, and selenium); vitamins (e.g., vitamin C, vitamin A, vitamin E, vitamin B 12, vitamin K, riboflavin, niacin, vitamin D, vitamin B6, folic acid, pyridoxine, thiamine, pantothenic acid, and biotin); buffering agents (e.g., sodium citrate, potassium phosphate, histidine, magnesium carbonate, magnesium bicarbonate, calcium carbonate, and calcium bicarbonate); preservatives (e.g., alpha-tocopherol, ascorbate, parabens, chlorobutanol, and phenol); binders (e.g., starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides); lubricants (e.g., magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil); dispersants (e.g., starch, alginic acid, polyvinylpyrrolidones, guar gum, kaolin, bentonite, purified wood cellulose, sodium starch glycolate, isoamorphous silicate, and microcrystalline cellulose); disintegrants (e.g., corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, tragacanth, sodium bicarbonate in combination with citric acid, and sodium bicarbonate in combination with tartaric acid); flavoring agents; sweeteners; and coloring agents.

In certain embodiments, the analytical method of the present invention quantifies two or more buffers or excipients selected from sucrose, hpβCD, sucrose, mannitol, histidine, sulfobutyl ether β-cyclodextrin, sodium phosphate, sodium citrate, potassium phosphate, trehalose, and Tris.

### HPLC

The analytical method of the present invention quantifies two or more buffers or excipients in a test sample by resolving the two or more buffers or excipients on a high performance liquid chromatography (HPLC) column. For example, in certain embodiments, the HPLC column is a pentafluorophenyl (PFP) column.

In some embodiments, the PFP column can facilitate fast, high-resolution separation of sample analytes at low backpressures. In some embodiments, the PFP column may be packed with particles having a diameter of about 0.8 μm to about 8.0 μm. For example, in some embodiments, the PFP column may be packed with particles having a diameter of about 1.7 μm, about 2.6 μm, or about 5.0 μm. In some embodiments, the PFP column may be packed with particles having a diameter of about 2.4 μm to about 2.6 μm. In certain embodiments, the PFP column may be packed with particles having a diameter of about 2.6 μm.

In some embodiments the particles of the PFP column may have pore diameters of about 60 Å to about 125 Å. In certain embodiments, the particles of the PFP column may have pore diameters of about 82 Å to about 102 Å. In certain embodiments, the PFP column may have pore diameters of about 100 Å.

As used herein, “size distribution” refers to a relative measure of particle diameter distribution. For example, a ratio of the particle diameter at 10% of the total size distribution and the particle diameter at 90% of the total size distribution can be used as a relative measure of the particle size distribution. The closer this ratio is to a value of 1, the more homogeneous the particle diameter distribution. In some embodiments, the particles of the PFP column may have a size distribution of less than or equal to about 1.5. For example, in some embodiments, the particles of the PFP column may have a size distribution of less than or equal to about 1.4, less than or equal to about 1.3, or less than or equal to about 1.2.

In some embodiments, the PFP column may have a diameter of about 2.0 mm to about 5.0 mm. For example, in some embodiments, the PFP column may have a diameter of about 2.1 mm, about 3 mm, or about 4.6 mm.

In some embodiments, the PFP column may have a length of about 10 mm to about 150 mm. For example, in some embodiments, the PFP column may have a length of about 10 mm, about 30 mm, about 50 mm, about 100 mm, or about 150 mm.

In some embodiments of the present invention, the two or more buffers or excipients are detected using an evaporative light scattering detector (ELSD). ELSDs use laser beams to measure the reflected light scattered to a photomultiplier, wherein the greater the size/mass of the particle, the greater the degree of light scattering.

In some embodiments, the ELSD is set at an evaporative temperature of about 10° C. to about 100° C. For example in some embodiments, the ELSD is set at an evaporative temperature of about 20° C. to about 100° C., about 30° C. to about 100° C., about 40° C. to about 100° C., about 50° C. to about 100° C., about 10° C. to about 90° C., about 20° C. to about 90° C., about 30° C. to about 90° C., about 40° C. to about 90° C., about 50° C. to about 90° C., about 10° C. to about 80° C., about 20° C. to about 80° C., about 30° C. to about 80° C., about 40° C. to about 80° C., about 50° C. to about 80° C., about 10° C. to about 70° C., about 20° C. to about 70° C., about 30° C. to about 70° C., about 40° C. to about 70° C., about 50° C. to about 70° C., about 10° C. to about 60° C., about 20° C. to about 60° C., about 30° C. to about 60° C., about 40° C. to about 60° C., or about 50° C. to about 60° C. In certain embodiments, the ELSD is set at an evaporative temperature of about 50° C.

In some embodiments, the ELSD is set at a pressure of about 10 psi to about 100 psi. For example, in some embodiments, the ELSD is set at a pressure of about 20 psi to about 100 psi, about 30 psi to about 100 psi, about 40 psi to about 100 psi, about 50 psi to about 100 psi, about 10 psi to about 90 psi, about 20 psi to about 90 psi, about 30 psi to about 90 psi, about 40 psi to about 90 psi, about 50 psi to about 90 psi, about 10 psi to about 80 psi, about 20 psi to about 80 psi, about 30 psi to about 80 psi, about 40 psi to about 80 psi, about 50 psi to about 80 psi, about 10 psi to about 70 psi, about 20 psi to about 70 psi, about 30 psi to about 70 psi, about 40 psi to about 70 psi, about 50 psi to about 70 psi, about 10 psi to about 60 psi, about 20 psi to about 60 psi, about 30 psi to about 60 psi, about 40 psi to about 60 psi, about 50 psi to about 60 psi. In certain embodiments, the ELSD is set at a pressure of about 50 psi.

In some embodiments the ELSD is set at a gain of 0.1 to 5. For example, in some embodiments, the ELSD is set to a gain of 0.1, 0.2, 0.3, 0.4, 0.5, 0.6, 0.7, 0.8, 0.9, 1.0, 1.5, 2, 2.5, 3, 3.5, 4, 4.5 or 5.

In some embodiments, the ELSD has a filter set at 0.1-1.0. For example, in some embodiments, the ELSD has a filter set at 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0.

In some embodiments, the ELSD is set at an evaporative temperature of about 10° C. to about 100° C. For example in some embodiments, the ELSD is set at an evaporative temperature of about 15° C. to about 100° C., about 20° C. to about 100° C., about 25° C. to about 100° C., about 30° C. to about 100° C., about 35° C. to about 100° C., about 40° C. to about 100° C., about 45° C. to about 100° C., about 50° C. to about 100° C., about 55° C. to about 100° C., about 10° C. to about 90° C., about 15° C. to about 90° C., about 20° C. to about 90° C., about 25° C. to about 90° C., about 30° C. to about 90° C., about 35° C. to about 90° C., about 40° C. to about 90° C., about 45° C. to about 90° C., about 50° C. to about 90° C., about 55° C. to about 90° C., about 10° C. to about 80° C., about 15° C. to about 80° C., about 20° C. to about 80° C., about 25° C. to about 80° C., about 30° C. to about 80° C., about 35° C. to about 80° C., about 40° C. to about 80° C., about 45° C. to about 80° C., about 50° C. to about 80° C., about 55° C. to about 80° C., about 10° C. to about 70° C., about 15° C. to about 70° C., about 20° C. to about 70° C., about 25° C. to about 70° C., about 30° C. to about 70° C., about 35° C. to about 70° C., about 40° C. to about 70° C., about 45° C. to about 70° C., about 50° C. to about 70° C., about 55° C. to about 70° C., about 10° C. to about 60° C., about 15° C. to about 60° C., about 20° C. to about 60° C., about 25° C. to about 60° C., about 30° C. to about 60° C., about 35° C. to about 60° C., about 40° C. to about 60° C., about 45° C. to about 60° C., about 50° C. to about 60° C., or about 55° C. to about 60° C.

In other embodiments, the two or more buffers or excipients are detected using a charged aerosol detector (CAD). CADs use high-voltage corona needles to charge nitrogen gas, which collides with analyte particles to produce charged particles.

In some embodiments, the CAD is set to a frequency of about 1 Hz to about 10 Hz. For example, in some embodiments, the CAD is set to a frequency of about 2 Hz to about 10 Hz, about 3 Hz to about 10 Hz, about 4 Hz to about 10 Hz, about 5 Hz to about 10 Hz, about 6 Hz to about 10 Hz, about 7 Hz to about 10 Hz, about 8 Hz to about 10 Hz, about 8 Hz to about 10 Hz, about 1 Hz to about 9 Hz, about 2 Hz to about 9 Hz, about 3 Hz to about 9 Hz, about 4 Hz to about 9 Hz, about 5 Hz to about 9 Hz, about 6 Hz to about 9 Hz, about 7 Hz to about 9 Hz, about 8 Hz to about 9 Hz, about 1 Hz to about 8 Hz, about 2 Hz to about 8 Hz, about 3 Hz to about 8 Hz, about 4 Hz to about 8 Hz, about 5 Hz to about 8 Hz, about 6 Hz to about 8 Hz, about 7 Hz to about 8 Hz, about 1 Hz to about 7 Hz, about 2 Hz to about 7 Hz, about 3 Hz to about 7 Hz, about 4 Hz to about 7 Hz, about 5 Hz to about 7 Hz, about 6 Hz to about 7 Hz, about 1 Hz to about 6 Hz, about 2 Hz to about 6 Hz, about 3 Hz to about 6 Hz, about 4 Hz to about 6 Hz, or about 5 Hz to about 6 Hz.

In some embodiments, the CAD has a filter set to about 1 second to about 10 seconds. For example, in some embodiments, the CAD has a filter set to about 2 seconds to about 10 seconds, about 3 seconds to about 10 seconds, about 4 seconds to about 10 seconds, about 5 seconds to about 10 seconds, about 6 seconds to about 10 seconds, about 7 seconds to about 10 seconds, about 8 seconds to about 10 seconds, about 8 seconds to about 10 seconds, about 1 second to about 9 seconds, about 2 seconds to about 9 seconds, about 3 seconds to about 9 seconds, about 4 seconds to about 9 seconds, about 5 seconds to about 9 seconds, about 6 seconds to about 9 seconds, about 7 seconds to about 9 seconds, about 8 seconds to about 9 seconds, about 1 second to about 8 seconds, about 2 seconds to about 8 seconds, about 3 seconds to about 8 seconds, about 4 seconds to about 8 seconds, about 5 seconds to about 8 seconds, about 6 seconds to about 8 seconds, about 7 seconds to about 8 seconds, about 1 second to about 7 seconds, about 2 seconds to about 7 seconds, about 3 seconds to about 7 seconds, about 4 seconds to about 7 seconds, about 5 seconds to about 7 seconds, about 6 seconds to about 7 seconds, about 1 second to about 6 seconds, about 2 seconds to about 6 seconds, about 3 seconds to about 6 seconds, about 4 seconds to about 6 seconds, or about 5 seconds to about 6 seconds.

In some embodiments, the CAD has a power function set to about 1.0 to about 2.0. For example, in some embodiments, the CAD has a power function set to about 1.1 to about 2.0, about 1.2 to about 2.0, about 1.3 to about 2.0, about 1.4 to about 2.0, about 1.5 to about 2.0, about 1.6 to about 2.0, about 1.0 to about 1.9, about 1.1 to about 1.9, about 1.2 to about 1.9, about 1.3 to about 1.9, about 1.4 to about 1.9, about 1.5 to about 1.9, about 1.6 to about 1.9, about 1.1 to about 1.8, about 1.2 to about 1.8, about 1.3 to about 1.8, about 1.4 to about 1.8, about 1.5 to about 1.8, or about 1.6 to about 1.8. In certain embodiments, the CAD has a power function set to about 1.78. In certain embodiments, the CAD has a power function set to about 1.68. In some embodiments the CAD has a power function that is set at a power function for the first two-thirds of an HPLC run that is different to the power function for the last third of the HPLC run. For example, in some embodiments, the CAD has a power function set to 1.71, 1.72, 1.73, 1.74, 1.75, 1.76, 1.77, 1.78, 1.79, or 1.80 for the first two-thirds of an HPLC run, and a power function set to 1.60, 1.61, 1.62, 1.63, 1.64, 1.65, 1.66, 1.67, 1.68, 1.69, or 1.70 for the last third of an HPLC run. In certain embodiments, the CAD has a power function set to 1.78 for the first two-thirds of an HPLC run, and a power function set to 1.68 for the last third of an HPLC run.

In still other embodiments, the two or more buffers or excipients are detected using a condensation nucleation light scattering detector (CNLSD). CNLSDs use water condensation to grow analyte particle sizes prior to subjecting the particles to laser beams for measuring reflected light scattered to a photomultiplier.

In some embodiments, the CNLSD is set at an evaporative temperature of about 10° C. to about 100° C. For example in some embodiments, the CNLSD is set at an evaporative temperature of about 20° C. to about 100° C., about 30° C. to about 100° C., about 40° C. to about 100° C., about 50° C. to about 100° C., about 10° C. to about 90° C., about 20° C. to about 90° C., about 30° C. to about 90° C., about 40° C. to about 90° C., about 50° C. to about 90° C., about 10° C. to about 80° C., about 20° C. to about 80° C., about 30° C. to about 80° C., about 40° C. to about 80° C., about 50° C. to about 80° C., about 10° C. to about 70° C., about 20° C. to about 70° C., about 30° C. to about 70° C., about 40° C. to about 70° C., about 50° C. to about 70° C., about 10° C. to about 60° C., about 20° C. to about 60° C., about 30° C. to about 60° C., about 40° C. to about 60° C., or about 50° C. to about 60° C. In certain embodiments, the CNLSD is set at an evaporative temperature of about 50° C.

In some embodiments, the CNLSD is set at a pressure of about 10 psi to about 100 psi. For example, in some embodiments, the CNLSD is set at a pressure of about 20 psi to about 100 psi, about 30 psi to about 100 psi, about 40 psi to about 100 psi, about 50 psi to about 100 psi, about 10 psi to about 90 psi, about 20 psi to about 90 psi, about 30 psi to about 90 psi, about 40 psi to about 90 psi, about 50 psi to about 90 psi, about 10 psi to about 80 psi, about 20 psi to about 80 psi, about 30 psi to about 80 psi, about 40 psi to about 80 psi, about 50 psi to about 80 psi, about 10 psi to about 70 psi, about 20 psi to about 70 psi, about 30 psi to about 70 psi, about 40 psi to about 70 psi, about 50 psi to about 70 psi, about 10 psi to about 60 psi, about 20 psi to about 60 psi, about 30 psi to about 60 psi, about 40 psi to about 60 psi, about 50 psi to about 60 psi. In certain embodiments, the CNLSD is set at a pressure of about 50 psi.

In some embodiments the CNLSD is set at a gain of 0.1 to 5. For example, in some embodiments, the CNLSD is set to a gain of 0.1, 0.2, 0.3, 0.4, 0.5, 0.6, 0.7, 0.8, 0.9, 1.0, 1.5, 2, 2.5, 3, 3.5, 4, 4.5 or 5.

In some embodiments, the CNLSD has a filter set at 0.1-1.0. For example, in some embodiments, the CNLSD has a filter set at 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0.

In some embodiments, the analytical method of the present invention quantifies two or more buffers or excipients in a test sample by HPLC using a mobile phase selected from 100% H₂O, 0.5% formic acid in H₂O, 0.05% trifluoroacetic acid in H₂O, and 100% acetonitrile. In some embodiments, the HPLC method uses more than one mobile phase: for example, 1, 2, 3, 4, 5, 7, 8, 9, or 10 mobile phases. In certain embodiments, the HPLC method uses two mobile phases: mobile phase A and mobile phase B. In certain embodiments, mobile phase A is selected from 100% H₂O, formic acid in H₂O, trifluoroacetic acid in H₂O, or combinations thereof, and mobile phase B comprises acetonitrile. In some embodiments the formic acid concentration is from about 0.5% to about 5%, e.g., about 0.5%, about 0.6%, about 0.7%, about 0.8%, about 0.9%, about 1.0%, about 1.5%, about 2.0%, about 2.5%, about 3.0%, about 3.5%, about 4.0%, about 4.5%, or about 5%. In some embodiments the trifluoroacetic acid concentration is from about 0.05% to about 0.5%, e.g., about 0.05%, about 0.06%, about 0.07%, about 0.08%, about 0.09%, about 0.1%, about 0.2%, about 0.3%, about 0.4%, or about 0.5%.

In some embodiments, the test sample is injected into the HPLC column at a volume of about 1 μl to about 50 μl. For example, in some embodiments, the HPLC injection volume is about 1 μl to about 45 μl, about 1 μl to about 40 μl, about 1 μl to about 35 μl, about 1 μl to about 30 μl, about 1 μl to about 25 μl, about 1 μl to about 20 about 1 μl to about 15 μl, about 1 μl to about 10 μl, about 5 μl to about 50 μl, about 5 μl to about 45 μl, about 5 μl to about 40 μl, about 5 μl to about 35 μl, about 5 μl to about 30 about 5 μl to about 25 μl, about 5 μl to about 20 μl, about 5 μl to about 15 μl, about 5 μl to about 10 μl, about 10 μl to about 50 μl, about 10 μl to about 45 μl, about 10 μl to about 40 μl, about 10 μl to about 35 μl, about 10 μl to about 30 μl, about 10 μl to about 25 μl, about 10 μl to about 20 μl, about 10 μl to about 15 μl, about 15 μl to about 50 μl, about 15 μl to about 45 μl, about 15 μl to about 40 μl, about 15 μl to about 35 μl, about 15 μl to about 30 μl, about 15 μl to about 25 μl, about 15 μl to about 20 μl, about 20 μl to about 50 μl, about 20 μl to about 45 μl, about 20 μl to about 40 μl, about 20 μl to about 35 μl, about 20 μl to about 30 μl, about 20 μl to about 25 μl, about 25 μl to about 50 μl, about 25 μl to about 45 μl, about 25 μl to about 40 μl, about 25 μl to about 35 μl, about 25 μl to about 30 μl, about 30 μl to about 50 μl, about 30 μl to about 45 μl, about 30 μl to about 40 μl, about 30 μl to about 35 μl, about 35 μl to about 50 about 35 μl to about 45 about 35 μl to about 40 about 40 μl to about 50 about 40 μl to about 45 or about 45 μl to about 50 μl. In certain embodiments, the injection volume is 1 μl, 2 μl, 3 μl, 4 μl, 5 μl, 6 μl, 7 μl, 8 μl, 9 or 10 μl.

In some embodiments the HPLC analysis comprises one or more than one steps. For example, in some embodiments, the HPLC analysis comprises 1, 2, 3, 4, 5, 6, 6, 8, 9, or 10 steps. In some embodiments, each step is for a duration of about 1.0 minute to about 10 minutes. For example, in some embodiments, each step is for a duration of about 1.5 minutes to about 10 minutes, about 2 minutes to about 10 minutes, about 2.5 minutes to about 10 minutes, about 3 minutes to about 10 minutes, about 3.5 minutes to about 10 minutes, about 4 minutes to about 10 minutes, about 4.5 minutes to about 10 minutes, about 5 minutes to about 10 minutes, about 5.5 minutes to about 10 minutes, about 6 minutes to about 10 minutes, about 6.5 minutes to about 10 minutes, about 7 minutes to about 10 minutes, about 7.5 minutes to about 10 minutes, about 8 minutes to about 10 minutes, about 8.5 minutes to about 10 minutes, about 9 minutes to about 10 minutes, about 9.5 minutes to about 10 minutes, about 1.0 minute to about 9 minutes, about 1.5 minutes to about 9 minutes, about 2 minutes to about 9 minutes, about 2.5 minutes to about 9 minutes, about 3 minutes to about 9 minutes, about 3.5 minutes to about 9 minutes, about 4 minutes to about 9 minutes, about 4.5 minutes to about 9 minutes, about 5 minutes to about 9 minutes, about 5.5 minutes to about 9 minutes, about 6 minutes to about 9 minutes, about 6.5 minutes to about 9 minutes, about 7 minutes to about 9 minutes, about 7.5 minutes to about 9 minutes, about 8 minutes to about 9 minutes, about 8.5 minutes to about 9 minutes, about 1.0 minute to about 8 minutes, about 1.5 minutes to about 8 minutes, about 2 minutes to about 8 minutes, about 2.5 minutes to about 8 minutes, about 3 minutes to about 8 minutes, about 3.5 minutes to about 8 minutes, about 4 minutes to about 8 minutes, about 4.5 minutes to about 8 minutes, about 5 minutes to about 8 minutes, about 5.5 minutes to about 8 minutes, about 6 minutes to about 8 minutes, about 6.5 minutes to about 8 minutes, about 7 minutes to about 8 minutes, about 7.5 minutes to about 8 minutes, about 1.0 minute to about 7 minutes, about 1.5 minutes to about 7 minutes, about 2 minutes to about 7 minutes, about 2.5 minutes to about 7 minutes, about 3 minutes to about 7 minutes, about 3.5 minutes to about 7 minutes, about 4 minutes to about 7 minutes, about 4.5 minutes to about 7 minutes, about 5 minutes to about 7 minutes, about 5.5 minutes to about 7 minutes, about 6 minutes to about 7 minutes, about 6.5 minutes to about 7 minutes, about 1.0 minute to about 6 minutes, about 1.5 minutes to about 6 minutes, about 2 minutes to about 6 minutes, about 2.5 minutes to about 6 minutes, about 3 minutes to about 6 minutes, about 3.5 minutes to about 6 minutes, about 4 minutes to about 6 minutes, about 4.5 minutes to about 6 minutes, about 5 minutes to about 6 minutes, about 5.5 minutes to about 6 minutes, about 1.0 minute to about 4 minutes, about 1.5 minutes to about 4 minutes, about 2 minutes to about 4 minutes, about 2.5 minutes to about 4 minutes, about 3 minutes to about 4 minutes, about 3.5 minutes to about 4 minutes, about 1.0 minute to about 3 minutes, about 1.5 minutes to about 3 minutes, about 2 minutes to about 3 minutes, about 2.5 minutes to about 3 minutes, about 1.0 minute to about 2 minutes, or about 1.5 minutes to about 2 minutes.

In some embodiments, each step of the HPLC analysis has a flow rate of about 0.1 ml/minute to about 5.0 ml/minute. For example, in some embodiments, each step has a flow rate of about 0.25 ml/minute to about 5.0 ml/minute, 0.5 ml/minute to about 5.0 ml/minute, 0.75 ml/minute to about 5.0 ml/minute, about 1.0 ml/minute to about 5.0 ml/minute, about 1.25 ml/minute to about 5.0 ml/minute, about 1.5 ml/minute to about 5.0 ml/minute, about 2.0 ml/minute to about 5.0 ml/minute, about 2.5 ml/minute to about 5.0 ml/minute, about 3.0 ml/minute to about 5.0 ml/minute, about 3.5 ml/minute to about 5.0 ml/minute, about 4.0 ml/minute to about 5.0 ml/minute, about 4.5 ml/minute to about 5.0 ml/minute, about 0.1 ml/minute to about 4.0 ml/minute, about 0.25 ml/minute to about 4.0 ml/minute, 0.5 ml/minute to about 4.0 ml/minute, 0.75 ml/minute to about 4.0 ml/minute, about 1.0 ml/minute to about 4.0 ml/minute, about 1.25 ml/minute to about 4.0 ml/minute, about 1.5 ml/minute to about 4.0 ml/minute, about 2.0 ml/minute to about 4.0 ml/minute, about 2.5 ml/minute to about 4.0 ml/minute, about 3.0 ml/minute to about 4.0 ml/minute, about 3.5 ml/minute to about 4.0 ml/minute, about 0.1 ml/minute to about 3.0 ml/minute, about 0.25 ml/minute to about 3.0 ml/minute, 0.5 ml/minute to about 3.0 ml/minute, 0.75 ml/minute to about 3.0 ml/minute, about 1.0 ml/minute to about 3.0 ml/minute, about 1.25 ml/minute to about 3.0 ml/minute, about 1.5 ml/minute to about 3.0 ml/minute, about 2.0 ml/minute to about 3.0 ml/minute, about 2.5 ml/minute to about 3.0 ml/minute, about 0.1 ml/minute to about 2.0 ml/minute, about 0.25 ml/minute to about 2.0 ml/minute, 0.5 ml/minute to about 2.0 ml/minute, 0.75 ml/minute to about 2.0 ml/minute, about 1.0 ml/minute to about 2.0 ml/minute, about 1.25 ml/minute to about 2.0 ml/minute, about 1.5 ml/minute to about 2.0 ml/minute, or about 0.5 ml/minute to about 1.0 ml/minute.

In some embodiments, each step of the HPLC analysis comprises: an equilibration step; a gradient change step, wherein the relative percentages of two or more different mobile phases are adjusted; a maintenance step, wherein the relative percentages of two or more different mobile phases are held constant; or a re-equilibration step.

In embodiments, where the HPLC analysis comprises using two mobile phases, each step may comprise a relative percentage of 100% mobile phase A, 0% mobile phase B; 90% mobile phase A, 10% mobile phase B; 80% mobile phase A, 20% mobile phase B; 70% mobile phase A, 30% mobile phase B; 60% mobile phase A, 40% mobile phase B; 50% mobile phase A, 50% mobile phase B; 40% mobile phase A, 60% mobile phase B; 30% mobile phase A, 70% mobile phase B; 20% mobile phase A, 80% mobile phase B; 10% mobile phase A, 90% mobile phase; or 0% mobile phase A, 100% mobile phase B.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: C18 and Anion-Exchange Columns Cannot Separate Excipients and/or Buffers in a Single Sample

Standard HPLC-based methods as known by persons of skill in the art are unable to resolve constituent excipients and/or buffers in a sample, wherein the sample comprises histidine, sucrose and mannitol, or tris, sucrose, and hpβCD.

### C18 Column HPLC and Detector Conditions

C18 column HPLC conditions are summarized in Table 1 below. Briefly, samples were loaded onto an Agilent Zorbax Eclipse Plus C18, 5 μm, 4.6×150 mm column. The column was equilibrated in 90% mobile phase A; 10% mobile phase B. At 6.0 min, the mobile phase was changed to 60% mobile phase A; 40% mobile phase B for 1 minute. From 7.0 min to 9.0 min, the column was maintained at 60% mobile phase A; 40% mobile phase B. At 9.0 min, the mobile phase was changed back to 90% mobile phase A; 10% mobile phase B for 1 minute and maintained at 90% mobile phase A; 10% mobile phase B for another 2 minutes to re-equilibrate the column. The flow rate was maintained at 1.0 ml/min throughout. The total cycle time was 12 minutes per run.

An Agilent 1200 series Evaporative Light Scattering Detector (ELSD) was used to acquire light scattering signals from excipients of interest. ELSD parameters are also summarized in Table 1 below. Briefly, evaporative temperature was set at 50° C. with a pressure of 50 psi. Gain and filter was set at 1 and 0.5, respectively.

TABLE 1

| Mobile Phase A | 100% Water | | | |
|---|---|---|---|---|
| Mobile Phase B | 100% Acetonitrile | | | |
| Injection Volume | 10 μL | | | |
| Gradient | Time | Flow rate | % A | %B |
| | (min) | (mL/min) | | |
| | 0 | 1.0 | 90.0 | 10.0 |
| | 6.0 | 1.0 | 90.0 | 10.0 |
| | 7.0 | 1.0 | 60.0 | 40.0 |
| | 9.0 | 1.0 | 60.0 | 40.0 |
| | 10.0 | 1.0 | 90.0 | 10.0 |
| | 12.0 | 1.0 | 90.0 | 10.0 |

| Column | Agilent Zorbax Eclipse Plus C18, 5 pm, 4.6 × 150 | |
|---|---|---|
| | mm | |
| Column Temperature | 50° C. ± 3° C. | |
| Autosampler Tray | 5° C. ± 3° C. | |
| Temperature | | |
| ELSD Settings | Evaporative Temperature | 50° C. |
| | Pressure | 50 psi (3.5 bar) |
| | Gain | 1 |
| | Filter | 0.5 |

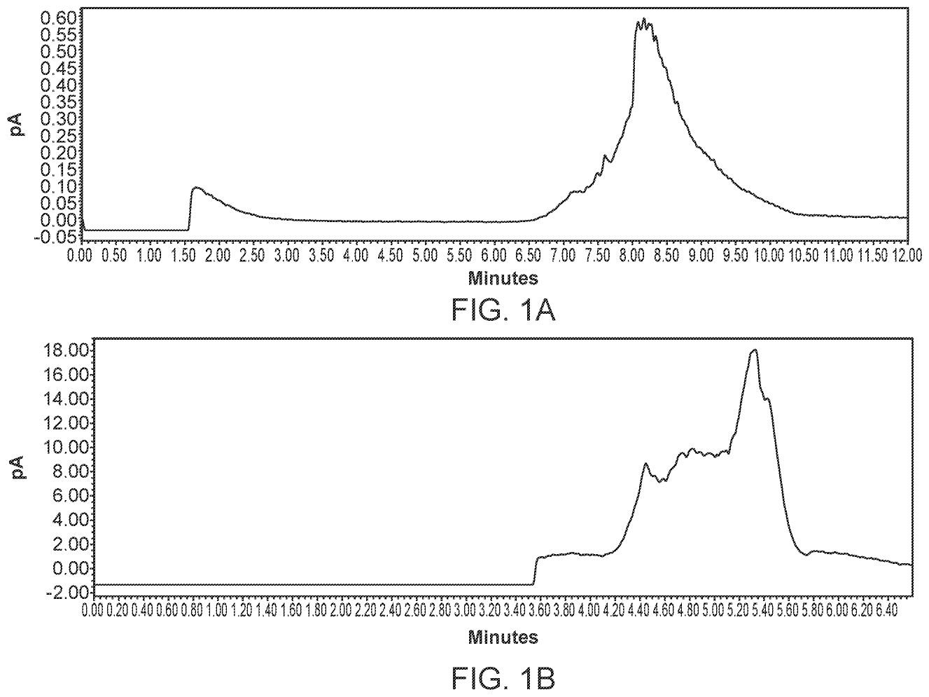

As shown in FIG. 1, a C18 column was not able to separate excipient and buffer peaks in a sample comprising histidine, sucrose, and mannitol (FIG. 1A), nor a sample comprising tris, sucrose, and hpβCD (FIG. 1B).

### Anion Exchange Column HPLC and Detector Conditions

Anion exchange column HPLC conditions are summarized in Table 2 below. Briefly, samples were loaded onto a Water Oasis MAX 2.1×20 mm, 30 μm column. The column was equilibrated in 90% mobile phase A; 10% mobile phase B for 1 minute. At 1.0 minute to 3.4 minutes, gradient changed to 80% mobile phase A; 20% mobile phase B. At 3.4 minutes to 3.5 minutes, gradient changed to 100% mobile phase B and was maintained for 1 minute. At 4.5 minutes, gradient changed back to 90% mobile phase A; 10% mobile phase B. At 4.6 minutes, gradient was maintained at 90% mobile phase A; 10% mobile phase B for 2 minutes. The flow rate was maintained at 1.0 ml/min throughout. The total cycle time was 6.6 minutes per run.

An Agilent 1200 series Evaporative Light Scattering Detector (ELSD) was used to acquire light scattering signals from excipients of interest. ELSD parameters are also summarized in Table 2 below. Briefly, evaporative temperature was set at 85° C. with a pressure of 3.5 bar±0.1. Gain and filter settings were variable and nominally at 4 and 4s, respectively.

TABLE 2

| Mobile Phase A | 2% Formic Acid in Water | | | |
|---|---|---|---|---|
| Mobile Phase B | 2% Formic Acid in Isopropanol | | | |
| Injection Volume | 40 μL | | | |
| Gradient | Time | Flow rate | % A | %B |
| | (min) | (mL/min) | | |
| | 0 | 1.0 | 90.0 | 10.0 |
| | 1.0 | 1.0 | 80.0 | 20.0 |
| | 3.4 | 1.0 | 80.0 | 20.0 |
| | 3.5 | 1.0 | 0.0 | 100.0 |
| | 4.5 | 1.0 | 0.0 | 100.0 |
| | 4.6 | 1.0 | 90.0 | 10.0 |
| | 6.6 | 1.0 | 90.0 | 10.0 |

| Column | Water Oasis MAX 2.1 × 20 mm, 30 μm | |
|---|---|---|
| Column Temperature | 60° C. ± 3° C. | |
| Autosampler Tray | 4° C. ± 3° C. | |
| Temperature | | |
| ELSD Settings | Evaporative Temperature | 85° C. |
| | Pressure | 3.5 bar ± 0.1 bar |
| | Gain | Variable (nominal at |
| | | 4) |
| | Filter | Variable (nominal at |
| | | 4 s) |

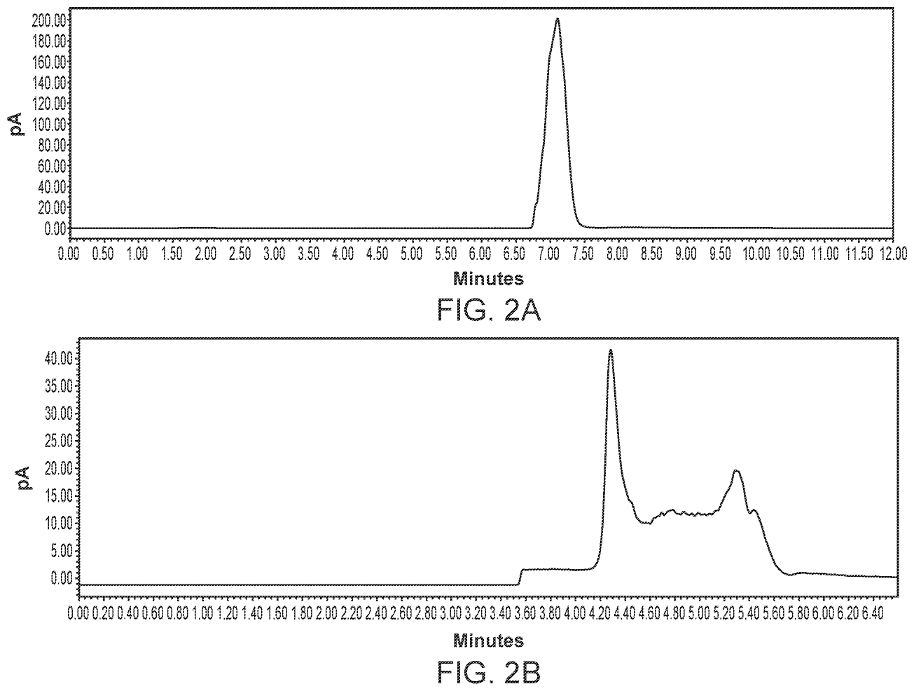

As shown in FIG. 2, an anion exchange column was not able to separate excipient and buffer peaks in a sample comprising histidine, sucrose, and mannitol (FIG. 2A), nor a sample comprising tris, sucrose, and hpβCD (FIG. 2B).

### Example 2: HPLC Quantification of HpβCD and Sucrose Using a PFP Column

An HPLC-based method was developed to separate and quantify hpβCD and sucrose in a test sample in a single assay.

### HPLC and Detector Conditions

HPLC conditions are summarized in Table 3 below. Briefly, a Kinetex Pentafluorophenyl (PFP) 2.6 μm 150×4.6 mm column was used to separate the excipients of interest. The column was equilibrated in 100% mobile phase A. At 3.5 min, the mobile phase gradient was changed to 60% mobile phase A; 40% mobile phase B. From 3.5 to 7.5 min, the gradient was gradually changed to 40% mobile phase A; 60% mobile phase B, then maintained at this gradient for 2.5 minutes. At 12 min, the mobile phase gradient was changed back to 100% mobile phase A. The flow rate started at 0.25 ml/minute then gradually raised to 1.0 ml/minute during excipients separation to achieve good separation between buffer species and sucrose. The sucrose and hpβCD were eluted between 4 and 10 minutes. The flow rate was reduced to 0.25 mg/mL at the end of run with 100% water in mobile phase for column re-equilibrium. The total cycle time was 15 minutes per run.

An Agilent 1200 series Evaporative Light Scattering Detector (ELSD) was used to acquire light scattering signals from excipients of interest. ELSD parameters are also summarized in Table 3 below. Briefly, evaporative temperature was set at 50° C. with a pressure of 50 psi. Gain and filter was set at 1 and 0.5, respectively.

TABLE 3

| Mobile Phase A | 100% Water | | | |
|---|---|---|---|---|
| Mobile Phase B | 100% Acetonitrile | | | |
| Injection Volume | 10 μL | | | |
| | Time | Flow rate | % A | % B |
| | (min) | (mL/min) | | |
| | 0.0 | 0.25 | 100.0 | 0.0 |
| | 3.0 | 0.25 | 100.0 | 0.0 |
| Gradient | 3.5 | 0.25 | 60.0 | 40.0 |
| | 7.5 | 1.00 | 40.0 | 60.0 |
| | 10.0 | 1.00 | 40.0 | 60.0 |
| | 12.0 | 1.00 | 100.0 | 0.0 |
| | 15.0 | 0.25 | 100.0 | 0.0 |

| Column | Kinetex Pentafluorophenyl (PFP) 2.6 um 150 × | |
|---|---|---|
| | 4.6 mm column | |
| Column Temperature | 25° C. ± 3° C. | |
| Autosampler Tray | 5° C. ± 3° C. | |
| Temperature | | |
| ELSD Settings | Evaporative Temperature | 50° C. |
| | Pressure | 50 psi (3.5 bar) |
| | Gain | 1 |
| | Filter | 0.5 |

### Calibration Curve Preparation and Test Sample Preparation

To quantify excipients, a calibration curve with excipients at known concentrations was prepared according to Table 4 below. The calibration curve range was established based on detector capability.

TABLE 4

| Target Concentration | Volume of 10% sucrose + | Volume |
|---|---|---|
| sucrose (%) + hpβCD | 10% hpβCD stock | of H₂O |
| (%) | solution (μL) | (μL) |
| 2.50 + 2.50 | 250 | 750 |
| 2.25 + 2.25 | 225 | 775 |
| 2.00 + 2.00 | 200 | 800 |
| 1.75 + 1.75 | 175 | 825 |
| 1.50 + 1.50 | 150 | 850 |

The test sample was injected into the HPLC column as neat if the expected concentration was within the calibration curve concentration range. If the test sample was expected to contain analytes at a concentration outside the range of the calibration curve, the test sample was diluted with H₂O to bring the concentration within the standard curve.

### HPLC Data Analysis

Peak of each analyte was integrated to obtain a peak area. A linear regression fit was performed for the peak area vs. concentration to calculate the slope (m) and intercept (b) of the calibration curve. This calibration curve was used to calculate the concentration of each analyte in the unknown sample using the equation below.

$$c ( \% ) = \frac{P e a k A r e a - b}{m}$$

where,
c=concentration of excipient in unknown sample
Peak Area=Peak area measured by integrating the excipient peak in the unknown sample chromatogram
b=y-intercept from the equation calculated using the calibration curve
m=slope from the equation calculated using the calibration curve

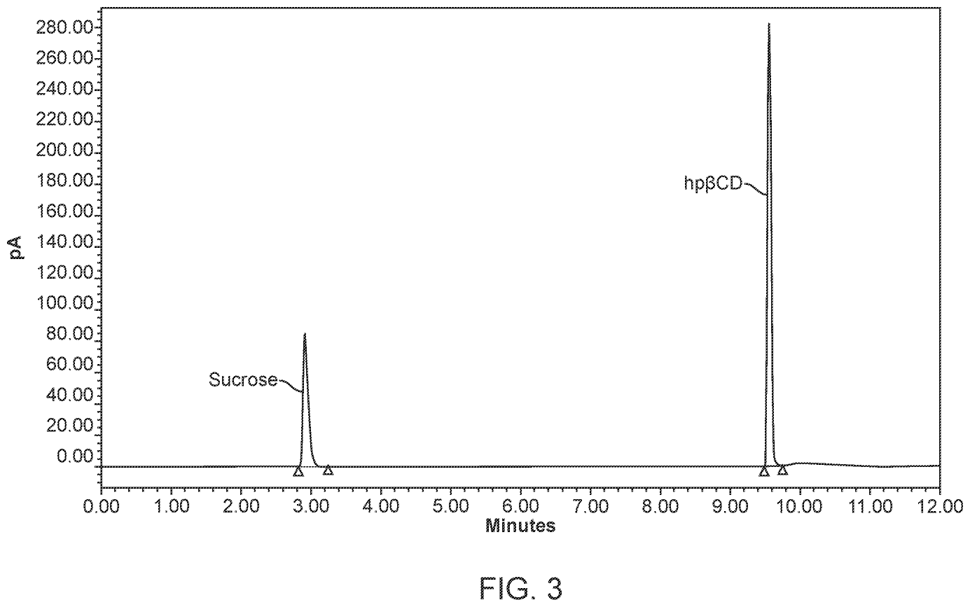

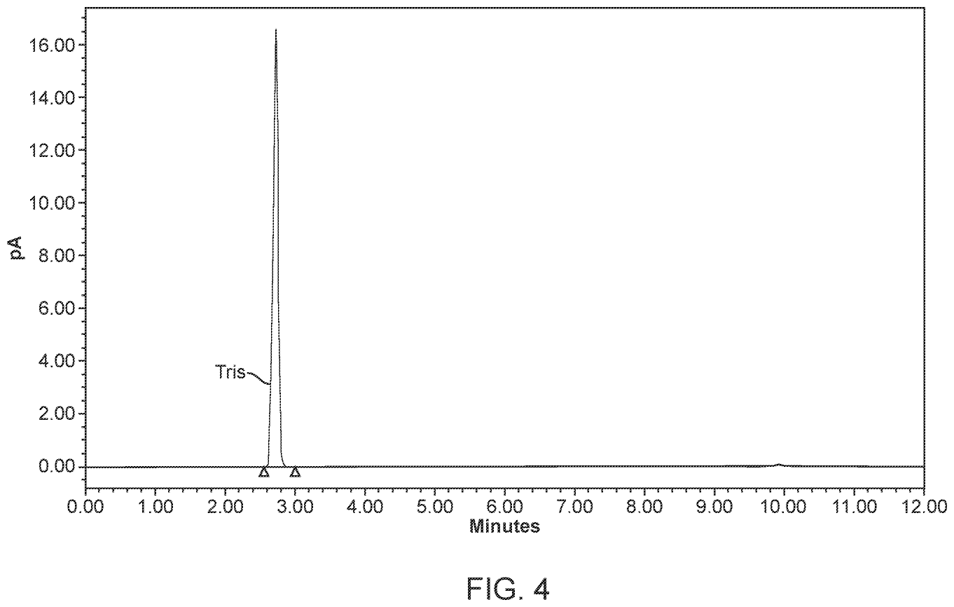

As shown in FIG. 3, an HPLC run using a PFP column under the conditions described in Table 3 resolves sucrose and hpβCD present in a single sample as defined peaks. Similarly, as shown in FIG. 4, an HPLC run using a PFP column under the same conditions clearly resolves Tris.

### Example 3: Quantification of Sucrose, HpβCD, and Other Excipients

Based on the work described in Example 2, a general HPLC method was developed to separate and quantify excipients using similar HPLC conditions but a Charged Aerosol Detector (CAD). This method can be used to quantify hpβCD and sucrose as well as several other excipients.

### HPLC and Detector Conditions

HPLC conditions are summarized in Table 5 below. Briefly, a Kinetex Pentafluorophenyl (PFP) 2.6 μm 150×4.6 mm column was used to separate the excipients of interest. The column was equilibrated in 100% mobile phase A, which consisted of water with 0.5% formic acid (FA). FA was used to improve peak shape of the buffer species (e.g., Tris buffer). From 4 to 6 min, the gradient was gradually changed to 40% mobile phase A; 60% mobile phase B. Then from 6 to 8 min, gradient was gradually returned to 100% mobile phase A. This gradient was maintained for additional 4 minutes to equilibrate the column. The flow rate was maintained at 0.5 ml/minute during the run. The sucrose was eluted around 3 minutes, hpβCD was eluted around 9.5 minutes, and Tris buffer was eluted around 2.8 minutes. The total cycle time was 12 minutes per run.

A ThermoFisher Corona™ Veo™ RS Charged Aerosol Detector (CAD) was used to acquire signals from excipients of interest. CAD parameters are also summarized in Table 5 below. Briefly, evaporative temperature was set at 35° C. with a frequency of 5 Hz and filter of 5 seconds. Power function was set to be 1.78 for the first 8 minutes and 1.68 for the last 4 minutes, respectively. Flow was diverted for the first 1.5 minutes directly to the waste.

TABLE 5

| Mobile Phase A | 0.5% FA in Water | | | |
|---|---|---|---|---|
| Mobile Phase B | 100% Acetonitrile | | | |
| Injection Volume | 4 μL | | | |
| Gradient | Time | Flow rate | % A | % B |
| | (min) | (mL/min) | | |
| | 0 | 0.5 | 100.0 | 0.0 |
| | 4.0 | 0.5 | 100.0 | 0.0 |
| | 6.0 | 0.5 | 40.0 | 60.0 |
| | 8.0 | 0.5 | 100.0 | 0.0 |
| | 12.0 | 0.5 | 100.0 | 0.0 |

| Column | Kinetex Pentafluorophenyl (PFP) 2.6 μm 150 × | |
|---|---|---|
| | 4.6 mm column | |
| Column Temperature | 25° C. ± 3° C. | |
| Autosampler Tray | 5° C. ± 3° C. | |
| Temperature | | |
| CAD Settings | Evaporative Temperature | 35° C. |
| | Hz | 5 |
| | Filter | 5.0 seconds |
| | Power Function (0-8 | 1.78 |
| | min) | |
| | Power Function (8-12 | 1.68 |
| | min) | |
| | Divert Flow | 0-1.5 min |

### Calibration Curve Preparation and Test Sample Preparation

To quantify excipients, a calibration curve with excipients at known concentrations was prepared according to Table 6 below. The calibration curve range was established based on detector capability. The CAD detector was capable of establishing a linear curve over a wider range compared to the ELSD detector in Example 2.

TABLE 6

| Target Concentration | Volume of 6% sucrose + | Volume |
|---|---|---|
| sucrose (%) + hpβCD | 6% hpβCD | of H₂O |
| (%) | stock solution (μL) | (μL) |
| 3.00 + 3.00 | 300 | 300 |
| 2.50 + 2.50 | 250 | 350 |
| 2.00 + 2.00 | 200 | 400 |
| 1.50 + 1.50 | 150 | 450 |
| 1,00 + 1.00 | 100 | 500 |
| 0.50 + 0.50 | 50 | 550 |

The test sample was injected into the HPLC column as neat if the expected concentration was within the calibration curve concentration range. If the sample was expected to contain analytes at a concentration outside the range of the calibration curve, the test sample was diluted with H₂O to bring the concentration within the standard curve.

### Data Analysis

Data analysis was performed as described in Example 1.

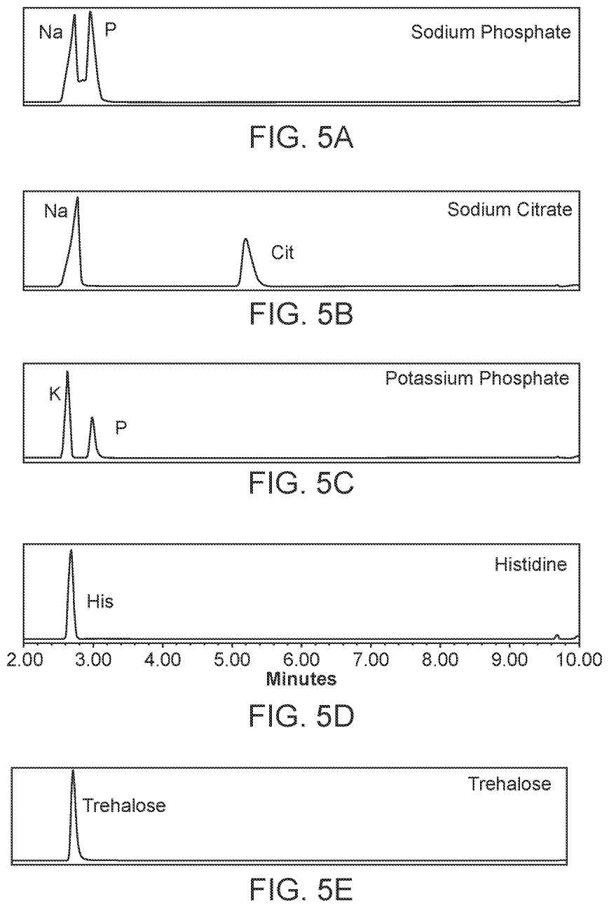

As shown in FIG. 5, an HPLC run using a PFP column under the conditions described in Table 5 was capable of resolving sodium phosphate (FIG. 5A), sodium citrate (FIG. 5B), potassium phosphate (FIG. 5C), histidine (FIG. 0.5D), and trehalose (FIG. 5E) present in a single sample as defined peaks.

### Example 4: Sucrose and Mannitol Quantification

An HPLC-based method was developed based on the work described in Example 2 to separate and quantify histidine, sucrose and mannitol content.

HPLC conditions are summarized in Table 6 below. Briefly, a Kinetex Pentafluorophenyl (PFP) 2.6 μm 150×4.6 mm column was used to separate the excipients of interest. The column was equilibrated in 100% mobile phase A consisted of water with 0.05% trifluoroacetic acid (TFA). TFA is needed to improve the separation of mannitol, sucrose and histidine. From 4 to 6 min, the gradient was gradually changed to 40% mobile phase A; 60% mobile phase B. Then from 6 to 8 min, gradient was returned to 100 mobile phase A gradually. This gradient was maintained for additional 4 minutes to equilibrate the column. The flow rate was maintained at 0.5 ml/minute during the run. The sucrose and mannitol were eluted between 2.5 and 3.5 minutes, and histidine was eluted between 3.5 to 4.5 minutes. The total cycle time was 12 minutes per run.

A ThermoFisher Corona™ Veo™ RS CAD was used to acquire signals from excipients of interest. CAD parameters were as described in Example 2.

TABLE 6

| Mobile Phase A | 0.05% TFA in Water | | | |
|---|---|---|---|---|
| Mobile Phase B | 100% Acetonitrile | | | |
| Injection Volume | 4 pL | | | |
| Gradient | Time | Flow rate | % A | %B |
| | (min) | (mL/min) | | |
| | 0 | 0.5 | 100.0 | 0.0 |
| | 4.0 | 0.5 | 100.0 | 0.0 |
| | 6.0 | 0.5 | 40.0 | 60.0 |
| | 8.0 | 0.5 | 100.0 | 0.0 |
| | 12.0 | 0.5 | 100.0 | 0.0 |

| Column | Kinetex Pentafluorophenyl (PFP) 2.6 pm 150 × | |
|---|---|---|
| | 4.6 mm column | |
| Column Temperature | 25° C. ± 3° C. | |
| Autosampler Tray | 5° C. ± 3° C. | |
| Temperature | | |
| CAD Settings | Evaporative Temperature | 35° C. |
| | Hz | 5 |
| | Filter | 5.0 seconds |
| | Power Function (0-8 | 1.78 |
| | min) | |
| | Power Function (8-12 | 1.68 |
| | min) | |
| | Divert Flow | 0-1.5 min |

### Calibration Curve Preparation and Test Sample Preparation

To quantify excipients, a calibration curve with excipients at known concentrations was prepared according to Table 7 below. The calibration curve range was established based on detector capability.

TABLE 7

| Target Concentration | Volume of 6% sucrose + 6% | Volume |
|---|---|---|
| sucrose (%) + mannitol | mannitol 60 mM histidine stock | of H₂O |
| (%) + histidine (mM) | solution (μL) | (μL) |
| 6.00 + 6.00 + 60 | 600 | 0 |
| 5.00 + 5.00 + 50 | 500 | 100 |
| 4.00 + 4.00 + 40 | 400 | 200 |
| 3.00 + 3.00 + 30 | 300 | 300 |
| 2.00 + 2.00 + 20 | 200 | 400 |
| 1.00 + 1.00 + 10 | 100 | 500 |

The test sample was injected into the HPLC column as neat if the expected concentration was within the calibration curve concentration range. If the sample was expected to contain analytes at a concentration outside the range of the calibration curve, the test sample was diluted with H₂O to bring the concentration within the standard curve.

### Data Analysis

Data analysis was performed as described in Example 1.

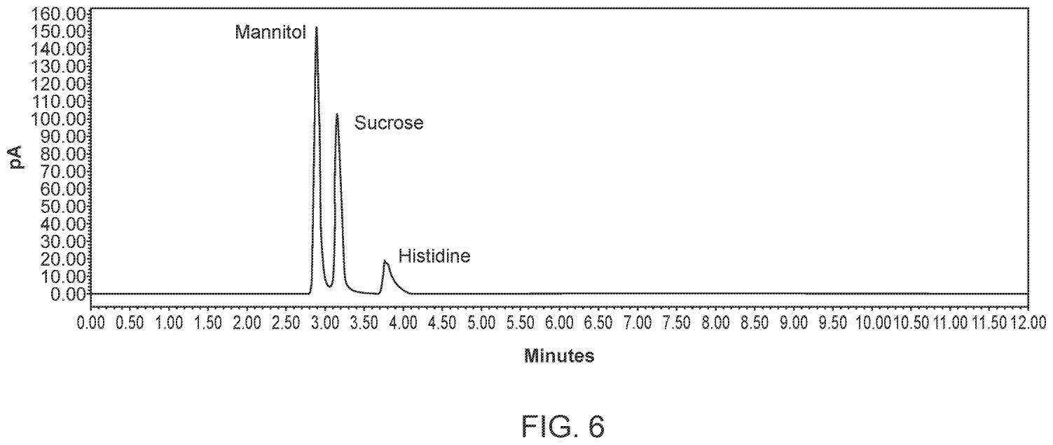

As shown in FIG. 6, an HPLC run using a PFP column under the conditions described in Table 6 was capable of resolving mannitol, sucrose, and histidine present in a single sample as defined peaks.

1. A method for analytically separating two or more buffers or excipients in a single assay, the method comprising:
performing chromatography on a test sample, the sample comprising the two or more buffers or excipients, on a pentafluorophenyl (PFP) high performance liquid chromatography (HPLC) column to separate the two or more buffers or excipients;
detecting the two or more separated buffers or excipients in the HPLC column effluent; and
generating a chromatogram having peaks corresponding to the separated two or more buffers or excipients.

2. The method according to claim 1, wherein the two or more buffers or excipients are selected from the group consisting of 2-hydroxypropyl-beta-cyclodextrin, sucrose, sodium phosphate, sodium citrate, potassium phosphate, histidine, trehalose, and mannitol.

3. The method according to claim 1 or 2, wherein the two or more buffers or excipients are sugars or sugar-based molecules.

4. The method according to claim 3, wherein the two or more buffers or excipients are sugars.

5. The method according to claim 3, wherein the two or more buffers or excipients are selected from the group consisting of 2-hydroxypropyl-beta-cyclodextrin, sucrose, trehalose, and mannitol.

6. The method according to any one of claims 1 to 5, further comprising:
obtaining standard calibration chromatographic data for the two or more excipients run on the same HPLC column; and
calculating a concentration or an amount of the two or more buffers or excipients in the test sample by determining from the chromatogram integrated peak areas of the two or more buffers or excipients and converting the integrated areas to a concentration or amount based on the obtained standard calibration chromatographic data.

7. The method according to claim 6, wherein the conversion includes a linear regression fit to the standard calibration chromatographic data.

8. The method according to any one of claims 1 to 7, wherein the two or more buffers or excipients are detected using an evaporative light scattering detector (ELSD).

9. The method according to claim 8, wherein the ELSD is set at an evaporative temperature of 40 to 70° C., a pressure of 30 to 70 psi, a gain of 0.5 to 2, and filter set at 0.5 to 1.

10. The method according to any one of claims 1 to 7, wherein the two or more buffers or excipients are detected using a charged aerosol detector (CAD).

11. The method according to claim 10, wherein the CAD is set at an evaporative temperature of 25 to 35° C., a frequency of 4 to 6 Hz, a filter set at 4 to 6 seconds, a power function set to 1.78 for the first two-thirds of an HPLC run, and a power function set to 1.68 for the last third of the HPLC run.

12. The method according to any one of claims 1 to 11, wherein the HPLC is run using mobile phase A and mobile phase B.

13. The method according to claim 12, wherein mobile phase A is selected from the group consisting of 100% H₂O, formic acid in H₂O, and trifluoroacetic acid in H₂O.

14. The method according to claim 13, wherein mobile phase A is selected from the group consisting of 0.5% formic acid in H₂O and 0.05% trifluoroacetic acid in H₂O.

15. The method according to any one of claims 12 to 14, wherein mobile phase B comprises acetonitrile.

16. The method according to claim 15, wherein mobile phase B is 100% acetonitrile.

17. The method according to any one of claims 12 to 15, wherein performing chromatography comprises an equilibration step having a flow of 100% mobile phase A through the HPLC column at a rate of 0.1 ml/minute to 1.0 ml/minute.

18. The method according to claim 17, wherein the equilibration step flow rate is 0.25 ml/minute.

19. The method according to claim 17, wherein the equilibration step flow rate is 0.5 ml/minute.

20. The method according to any one of claims 17 to 19, wherein the equilibration step is between 0.5 minutes and 10 minutes.

21. The method according to claim 20, wherein the equilibration step is 3.0 minutes.

22. The method according to claim 20, wherein the equilibration step is 4.0 minutes.

23. The method according to any one of claims 12 to 22, wherein performing chromatography comprises a gradient change step flow of 60% mobile phase A and 40% mobile phase B through the HPLC column.

24. The method according to any one of claims 12 to 22, wherein performing chromatography comprises a gradient change step flow of 40% mobile phase A and 60% mobile phase B through the HPLC column.

25. The method according to any one of claims 12 to 22, wherein performing chromatography comprises a gradient change step flow of 100% mobile phase A through the HPLC column.

26. The method according to any one of claims 23 to 25, wherein the gradient change step flow rate is between 0.1 ml/minute to 1.0 ml/minute.

27. The method according to claim 26, wherein the gradient change step flow rate is 0.25 ml/minute.

28. The method according to claim 26, wherein the gradient change step flow rate is 0.5 ml/minute.

29. The method according to any one of claims 23 to 28, wherein the gradient change step is between 0.5 minutes and 10 minutes.

30. The method according to claim 29, wherein the gradient change step is 0.5 minutes.

31. The method according to claim 29, wherein the gradient change step is 2.0 minutes.

32. The method according to claim 29, wherein the gradient change step is 4.0 minutes.

33. The method according to any one of claims 12 to 32, wherein performing chromatography comprises a maintenance step flow of 40% mobile phase A and 60% mobile phase B through the HPLC column.

34. The method according to any one of claims 12 to 32, wherein performing chromatography comprises a maintenance step flow of 100% mobile phase A through the HPLC column.

35. The method according to claim 33 or 34, wherein the maintenance step flow rate is between 0.1 ml/minute and 1.0 ml/minute.

36. The method according to claim 35, wherein the maintenance step flow rate is 0.5 ml/minute.

37. The method according to claim 35, wherein the maintenance step flow rate is 1.0 ml/minute.

38. The method according to any one of claims 33 to 37, wherein the maintenance step is between 0.5 minutes and 10 minutes.

39. The method according to claim 38, wherein the maintenance step is 2.5 minutes.

40. The method according to claim 38, wherein the maintenance step is 4.0 minutes.

41. The method according to any one of claims 12 to 40, wherein performing chromatography comprises a re-equilibration step having a flow of 100% mobile phase A through the HPLC column at a rate of 0.1 ml/minute to 1.0 ml/minute.

42. The method according to claim 41, wherein the re-equilibration step flow rate is 0.25 ml/minute.

43. The method according to claim 41 or 42, wherein the re-equilibration step is between 0.5 minutes and 10 minutes.

44. The method according to claim 43, wherein the re-equilibration step is 3.0 minutes.

45. The method according to any one of claims 12 to 15, wherein performing chromatography comprises: (i) equilibration with 100% mobile phase A at a flow rate of 0.25 ml/minute for 3.0 minutes; (ii) gradient change to 60% mobile phase A and 40% mobile phase B at a flow rate of 0.25 ml/minute for 0.5 minutes; (iii) gradient change to 40% mobile phase A and 60% mobile phase B at a flow rate of 1.0 ml/minute for 4.0 minutes; (iv) maintenance at 40% mobile phase A and 60% mobile phase B at a flow rate of 1.0 ml/minute for 2.5 minutes; (v) gradient change to 100% mobile phase A at a flow rate of 1.0 ml/minute for 2 minutes; and (vi) re-equilibration with 100% mobile phase A at a flow rate of 0.25 ml/minute for 3.0 minutes.

46. The method according to any one of claims 12 to 15, wherein performing chromatography comprises: (i) equilibration with 100% mobile phase A at a flow rate of 0.5 ml/minute for 4.0 minutes; (ii) gradient change to 40% mobile phase A and 60% mobile phase B at a flow rate of 0.5 ml/minute for 2.0 minutes; (iii) gradient change to 100% mobile phase A at a flow rate of 0.5 ml/minute for 2.0 minutes; and (iv) maintenance at 100% mobile phase A at a flow rate of 0.5 ml/minute for 4 minutes.

47. The method according to any one of claims 12 to 15, wherein performing chromatography comprises: (i) equilibration with 100% mobile phase A at a flow rate of 0.5 ml/minute for 4.0 minutes; (ii) gradient change to 40% mobile phase A and 60% mobile phase B at a flow rate of 0.5 ml/minute for 2.0 minutes; (iii) gradient change to 100% mobile phase A at a flow rate of 0.5 ml/minute for 2.0 minutes; and (iv) maintenance at 100% mobile phase A at a flow rate of 0.5 ml/minute for 4 minutes.

48. The method according to any one of claims 1 to 47, wherein 1 μl to 100 μl of the test sample is injected into the HPLC column.

49. The method according to claim 48, wherein 10 μl of the test sample is injected into the HPLC column.

50. The method according to claim 48, wherein 4 μl of the test sample is injected into the HPLC column.

51. The method according to any one of claims 1 to 50, wherein the PFP HPLC column is a 2.6 μm 150×4.6 mm column.
